(19) United States
(12) Patent Application Publication
(10) Pub. No.: US 2016/0338881 A1
(43) Pub. Date: Nov. 24, 2016

(54) StapHresh Female Performance Panty

(71) Applicant: Laquinta Hyppolite, Pembroke Pines, FL (US)

(72) Inventor: Laquinta Hyppolite, Pembroke Pines, FL (US)

(21) Appl. No.: 14/719,963

(22) Filed: May 22, 2015

(51) Int. Cl. A61F 13/472; A61F 13/15

(52) U.S. Cl. CPC A61F 13/472; A61F 13/15268; A61F 2013/15276; A61F 2013/4708

(57) ABSTRACT

An antimicrobial article comprising of antimicrobial panty and an absorbent pad designed specifically for women, for absorbing body fluids in the form of an absorbent pad comprising a fluid storage region and a fluid receiving region to provide support, absorb moisture within the covered areas, reduce vaginal odor, decrease risk of skin infection and to provide high quality of feminine care to women and girls wherein the antimicrobial article in the form of an absorbent pad comprises of composite textile fabric including muslin cotton crouch pocket, polyester and spandex, enhancing the transport of the moisture like sweat or other body fluids away from the skin.

### CROSS REFERENCE TO RELATED APPLICATIONS

Not applicable.

### FEDERALLY SPONSORED RESEARCH AND DEVELOPMENT

Not applicable,

### MICROFICHE APPENDIX

Not applicable.

### BACKGROUND OF THE INVENTION

(1) Field of the Invention

The invention relates to a composite anti-microbial undergarment textile fabric and more particularly to a composite fabric comprising spandex, polyester and muslin cotton. Together, layers of these different materials act to move urine or other fluids away from the skin and through an undergarment made with the composite fabric. The invention also relates to disposable absorbent articles, in the form of pad, which exhibit cloth like features for improved, comfort and is anti-infectious in nature.

(2) Background of Invention

Disposable absorbent devices for the absorption of human exudates are widely used. These disposable devices typically have a compressed mass of absorbent formed into the desired shape, which is typically dictated by the intended consumer use.

There exists in the female body a complex process which maintains the vagina and physiologically related areas in a healthy state. In a female between the, age of menarche and menopause, the normal vagina provides an ecosystem for a variety of micro-organisms. Bacteria are the predominant type of micro-organisms present in the vagina; most women harbor about 10⁹ bacteria per gram of vaginal exudate. The bacterial flora of the vagina is comprised of both aerobic and anaerobic bacteria and some kinds of viruses too, These latter micro-organisms are generally associated with vaginitis or venereal disease, although they may be present in low numbers without causing symptoms.

Physiological, social and idiosyncratic factors affect the quantity and species of bacteria present in the vagina. Physiological factors include age, days of the menstrual cycle, and pregnancy. Social and idiosyncratic factors include methods of birth control, sexual practices, and systemic diseases (e.g., diabetes), and medication. Bacterial proteins and metabolic products produced in the vagina can affect other micro-organisms and the human host.

A significant problem for females generally is the ability to keep their undergarments infectious free, sweat absorbing and odoured during their exercise regimen and other activities as well. Most conventional undergarments textile fabrics are likely to result in substantial disclosure of sweat between the wearer's skin and the undergarment. When saturation of the undergarment takes place, accumulated sweat wets the body of the garment wearer such that the person begins to feel uncomfortable.

Adult size diaper materials besides being bulky, and uncomfortable cannot be laundered or reused, Where a super absorbent material is used, the material is usually in the form of powder. While commercially available super-absorbent powders offer a range of water-absorbent offer a range of water-absorbent characteristics, they can be difficult to incorporate into absorbent products. Furthermore, there is a tendency for them to migrate from their location, special equipment is often required to handle them and they must be kept in place by gluing, fusing or lamination to support structure. Also the gels formed from powders and water has little integrity and are, therefore difficult to contain within a structure when subject to pressure.

Accordingly, it would be desirable to provide a composite material which overcomes the above disadvantages and which helps transport moisture such as sweat away from the skin of the wearer.

### BRIEF SUMMARY OF THE INVENTION

Generally speaking, in accordance with the invention, a composite textile anti-microbial fabric for moving moisture like sweat or other body fluids away from the skin and retaining it is provided.

Accordingly, it is an object of the present invention to provide an improved composite material for enhancing the transport of body fluids like sweat away from the skin.

A further object of the present invention is to provide a composite material which includes a super absorbent material.

Yet another object of the present invention is to provide an improved composite material for an undergarment in which the undergarment has the drape-ability and feel of a conventional undergarment and can be laundered and reused in a conventional manner.

It is another object of the present invention to provide a feminine care absorbent article comprising an absorbent structure configured for disposition adjacent a female wearer's vaginal region to absorb bodily fluids discharged by the wearer.

Other objects and advantages of the present invention and modifications thereof will become apparent to persons, skilled in art without departure from the inventive concepts defined in the appended claims.

### BRIEF DESCRIPTION OF THE SEVERAL VIEWS OF THE DRAWINGS

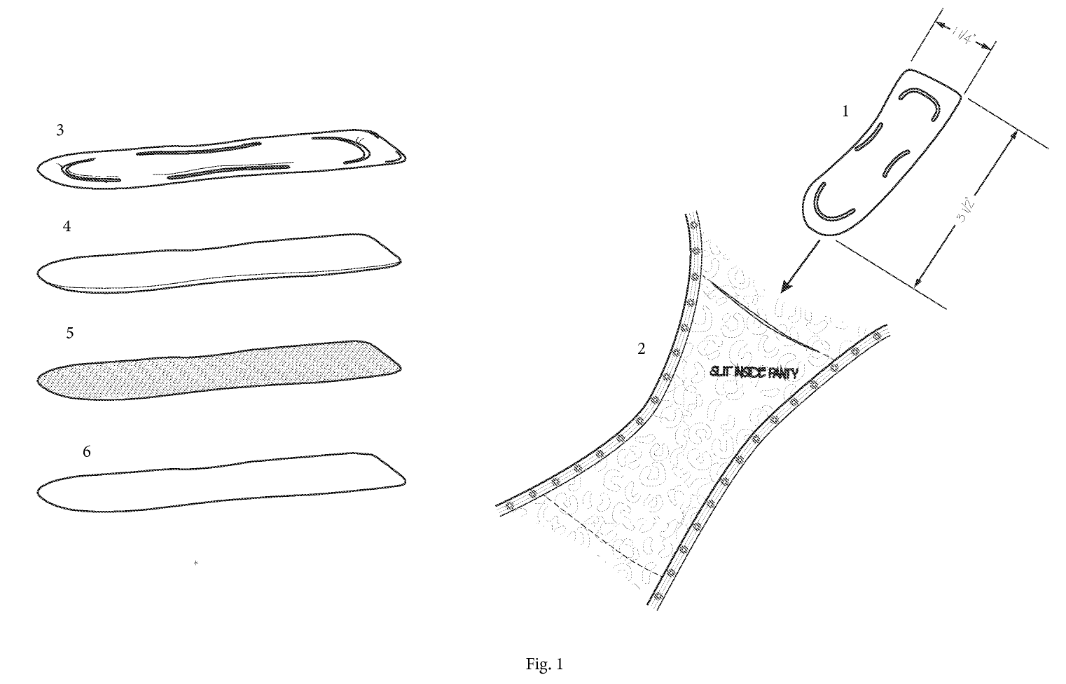

To further clarify various aspects of some example embodiments of the present invention, a more particular description of the invention will be rendered by reference to specific embodiments thereof which are illustrated in the appended drawing. It is appreciated that the drawing depicts only illustrated embodiments of the invention and are therefore not to be considered limiting of its scope. The invention will be described and explained with additional specificity and detail through the use of the accompanying drawing in which: FIG. 1 is a perspective view of the present invention.

### DETAILED DESCRIPTION OF THE INVENTION

The absorbent product of the present invention FIG. 1, provides an absorbent article 1 which is designed to adhere to the body of a wearer 2 in the area of the body of the wearer which may need bodily fluids absorbed. In one particular use of the absorbent article, the absorbent article is attached to the body of female wearer to or around the vulva region of the body. By “to or around the vulva region”, it is meant adjacent regions of the body of a female including the pubic region and the perinea region. When applied to or around the vulva region of the female body, the absorbent article may be used as a panty-liner, sanitary napkin etc. As an article, the absorbent article serves to protect the outer clothing of the wearer from bodily discharges from the vulva region of the wearer's body. The absorbent article also serves to protect the sensitive skin and body features of the vulva region from roughness of the outer clothing, thereby preventing or alleviating irritation to the sensitive skin and body features of the vulva region,

The invention relates to an absorbent article. it particularly concerned with an absorbent article, or example in the form of pad. The invention will be so described below. However, the invention is of more general applicability in relation to the absorption of body fluids.

An absorbent pad, or other article for use should desirably:

(a) Be thin, and with a shape fitting well into the underpants, to provide discretion when worn under normal clothing.

(b) Be absorbent enough to handle large quantities of body fluids.

(c) Absorb rapidly enough.

The absorbent pad having a unique circular bottom and rectangular top shaped that provides added comfort and dryness. The ChemiPad is designed to securely fit in the crouch pocket of the StapHresh panty. The length of the pad is 3½ inches long and 1¼ inches wide. It is smaller than the usual size pad or panty liner. The absorbent fabric pad has four layers: non-woven layer to absorb moisture 3, a woven cloth with vitamin C, baking soda, and chamomile for anti-bacteria, odor reducing, and soothing support 4, an air-laid paper 5; and a muslin cotton layer 6.

According to one aspect of present invention, there is provided an article for absorbing fluid, which comprises a fluid storage region and a fluid receiving region adapted to release fluid to fluid storage region.

In other preferred embodiment, the present invention provides an anti-microbial panty that reduces the risk of female skin and vaginal infections. Also has an insertible pad into a slip/casing in the middle that attracts odor and moisture.

In another preferred embodiment, the present invention provides support, absorb moisture within the covered areas, reduce vaginal odor, decrease risk of skin infection and secure high quality of feminine care for women and girls.

In yet another preferred embodiment, the present invention decreases the risk of body disorders such as skin and vaginal infections by eliminating the use of body sprays, powders, and improper use of deodorant, creams and vaginal douche.

In another preferred embodiment, the present invention saves time, money and prevents medical cost associated with bacterial or fungal infections.

In another preferred embodiment, the present invention provides universal extra small, small, medium, large and extra-large sizes absorbing article made of 100% muslin cotton pocket, 83% polyester and 17% spandex.

In another preferred embodiment, the present invention provides a crouch pocket that is designed to support the absorbent anti-odor, herbal and vitamin infused pad.

In another preferred embodiment of the present invention, the pad size is 3½ inches long and 1¼ inches wide.

Although specific embodiments have been illustrated and described herein, it will be appreciated by those of ordinary skill in the art that any arrangement, which is calculated to achieve the same purpose, may be substituted for the specific embodiment shown. This application is intended to cover any adaptations or variations of the present invention, Therefore, it is manifestly intended that this invention be limited only by the claims and the equivalents thereof.

Although the invention has been explained in relation to its preferred embodiment, it is to be understood that many other possible modifications and variations can be made without departing from the spirit and scope of the invention.

I claim:

1. An anti-microbial undergarment for women enhancing the transport of the moisture of body fluids away from the skin comprising:
(a) An antimicrobial panty having a crouch muslin cotton pocket; and
(b) absorbent pad.

2. The anti-microbial undergarment of claim wherein said antimicrobial panty comprises of a composite of a super absorbent rewashable and reusable textile fabric.

3. The anti-microbial undergarment of claim 1, wherein said absorbent pad comprises of an absorbent structure configured for disposition adjacent a female wearer's vaginal region to absorb bodily fluids discharged by the wearer.

4. The anti-microbial undergarment of claim 1, wherein said absorbent pad comprises of a fluid storage region and a fluid receiving region adapted to release fluid to fluid storage region.

5. The anti-microbial undergarment of claim 1, wherein said absorbent pad comprise
non-woven layer to absorb moisture:
a woven cloth with vitamin C, baking soda and chamomile for anti-bacteria, odor reducing, and soothing support;
an air-laid paper; and
a muslin cotton layer.

6. The anti-microbial undergarment of claim 1, wherein said absorbent pad size is 3½ inches long and 1¼ inches wide.

7. The anti-microbial undergarment of claim 1, wherein said antimicrobial panty comprises of multilayer fabric including 100% muslin cotton crouch pocket, 83% polyester and 17% spandex.

8. The anti-microbial undergarment of claim 1, wherein said cotton crouch pocket is designed to support the absorbent anti-odor, herbal and vitamin infused pad.
